(19) United States
(12) Patent Application Publication
(10) Pub. No.: US 2016/0175150 A1
(43) Pub. Date: Jun. 23, 2016

(54) ULTRASONIC HANDPIECE WITH MULTIPLE DRIVERS

(71) Applicant: SURGICAL DESIGN CORPORATION, Armonk, NY (US)

(72) Inventor: William Banko, Armonk, NY (US)

(21) Appl. No.: 14/575,466

(22) Filed: Dec. 18, 2014

(51) Int. Cl. A61F 9/007; A61B 17/32

(52) U.S. Cl. CPC A61F 9/00745; A61B 17/320068

(57) ABSTRACT

A surgical handpiece has at least two working tips and at least two ultrasonic drivers. Each of the ultrasonic drivers creates longitudinal mechanical vibration. At least two connecting pieces connect each of the ultrasonic drivers to one separate working tip so that the mechanical vibration of one driver is applied to one tip independent of the application of the mechanical vibration of the other driver applied to the other tip. A control system independently controls the amplitude of the mechanical vibration from each of the ultrasonic drivers. In one embodiment the drivers are both in a single housing, and optionally a lever is provided on the handpiece to operate in conjunction with one or both of the tips. In another embodiment, the drivers are in separate housings that can be pivoted with respect to each other.

### TECHNICAL FIELD

The present invention is generally directed to an ultrasonic handpiece with at least two ultrasonic drivers that operate two tips. The handpiece can be used in connection with endoscopic surgery or eye surgery, e.g., the removal of cataracts by phacoemulsification.

### BACKGROUND OF THE INVENTION

The use of ultrasonic instruments in surgical applications is well known. One widely used type of instrument is an ultrasonic handpiece that is used in ophthalmic applications, such as in the removal of cataracts from the eye by phacoemulsification. For example, U.S. Pat. No. 4,428,748 of Peyman et al. discloses a surgical system and apparatus powered by a control central. The system includes a handpiece with an ultrasonic motor for driving a needle or other instrument with ultrasound.

Similarly, U.S. Pat. No. 4,504,264 of Kelman discloses a handpiece with a housing of, for example, plastic or metal, within which is supported a transducer for generating mechanical vibrations upon excitation with an alternating-current electrical signal. The transducer is shown as a magnetostrictive transducer with an electrical coil wound about a stack of metal laminations so that longitudinal mechanical vibrations are produced. The patent notes that the transducer can also be of the piezoelectric type. There is a connecting body of, for example, titanium, having a reduced diameter distal end portion, which also can be an attached separate portion. The connecting body forms an acoustic impedance transformer for conveying the longitudinal vibrations of the transducer for application to an operative tool or working tip connected to the distal end of the connecting body. The handpiece also has electrical input terminals for applying a suitable electrical signal to the magnetostrictive transducer.

In the Kelman device there is sleeve around the tip that forms a first fluid passage between the tip and the sleeve for an infusion/irrigation fluid. An inlet is provided on the housing or sleeve for supplying the irrigation fluid to the passage from a fluid supply, e.g., a bag of saline solution. A passage is formed through the connecting body that is in communication with a central passage of the work tip. An outlet on the housing or sleeve receives a suction (aspiration) force that is applied to the passage in the connecting body and the central passage in the work tip. A chamber is formed on the body and the housing, with which the aspiration force from the outlet communicates. Thus, the aspiration force is from the source (e.g., a suction pump), into the chamber, through the passage in the connecting body and the passage in the work tip. Tissue that is emulsified by the work tip is aspirated from the operating site by the aspiration flow force. In particular, saline solution introduced into the eye through the fluid passage and tissue displaced by the vibration force of the tip, is drawn into the distal end of passage and passes out of the handpiece through an outlet.

Instruments of the type described above are often used in cataract surgery in which the eye lens is removed from the eye capsule and an intra-ocular lens (IOL) is then implanted. In such a procedure before the IOL is implanted it has been found to be desirable to cleanup lens particles and lens epithelial cells (LEC's) in the capsular bag of the eye, i.e. to remove them. Performing this procedure provides a more stable and long-term fixation for certain types of IOL's in the capsular bag. One manner of accomplishing the cleanup is to use a combination of irrigation of the capsular bag interior with a liquid together with the application of low power ultrasonic energy. This dislodges the unwanted cells and particles so that they can be removed from the capsular bag by the aspiration fluid flow.

An ultrasonic operation apparatus is disclosed by U.S. Pat. No. 6,053,906 of Honda et al. The Honda apparatus comprises a drive circuit unit connected to a handpiece. The handpiece contains an ultrasonic oscillator for causing ultrasonic oscillation and a probe for transmitting the ultrasonic oscillation from the ultrasonic oscillator to a treatment section. A holding portion is supported on the distal end of a sheath, and is provided in the treatment section. The holding portion can move toward and away from a distal end portion of the probe so as to grip human tissue.

As shown in U.S. Pat. No. 7,083,589 of the present inventor (William Banko), the surgical instrument may be provided with a coupler body located between the connecting body and the work tip. In such a case the aspiration fluid flow is provided from the work tip aspiration passage through the coupler to an outlet without coming into contact with the interior of the connecting body. Irrigation fluid can be provided through a portion of the housing that surrounds the proximal part of the work tip so as to form a chamber which is in communication with a separate passage in the work tip. The coupler is detachably connected to the connecting body. This allows the removal of the work tip, which becomes a single use part, so that the rest of the instrument can be reused by replacing the work tip without having to sterilize the connecting body. However, the portion of the housing surrounding the work tip and which forms the chamber for irrigation fluid, also needs to be replaced in this design.

U.S. Pat. No. 8,663,220 of Wiener et al. discloses a surgical instrument that can supply mechanical energy and electrical energy to an end effector of the surgical instrument. The surgical instrument may comprise a clamp, or jaw, which can be moved into a closed position to hold tissue. The mechanical energy comes from an ultrasonic generator module.

Each of the instruments described above-has a single transducer that provides a vibration force for breaking up tissue. However, there also exist ultrasonic tools that have other functions. For example, Synthes GmbH of Oberdorf, Germany distributes an ultrasonic handpiece with various cutting pieces, e.g., a bone cutter.

Moog Medical Devices Group of Salt Lake City Utah makes ultrasonic devices used in phacoemulsification as described above. However, it also makes similar devices for use in neurosurgery, orthopedic surgery and liposuction. Further, devices with a stationary tip pivotally coupled to a vibrated tip can be used as scissors, saws and coagulators. For example, U.S. Published Application No. 2007/0282333 of Fortson et al. discloses an ultrasonic clamp coagulator assembly that is configured to permit selective cutting, coagulation, and clamping of tissue during surgical procedure.

Further, there are ultrasonic surgical devices that are equipped with multiple ultrasonic vibrating end portions. For example, U.S. Pat. No. 8,142,457 of Lafontaine discloses methods and devices for performing intravascular endarterectomy. The device has a “paddle” that is used to remove plaque from vessel walls. The paddles include ultrasonic imaging paddles, ultrasonic vibrating paddles, and mechanically vibrating paddles. In one embodiment of Lafontaine (FIG. 3A), a pair of paddles are used. However, these paddles are structurally and functionally identical with each other and are not enclosed inside one housing. Accordingly, in case a different operation is desired, one must exchange the paddle.

Since the frequency of an ultrasonic driver or transducer is set by its length, each of the devices described above is limited in use once it is constructed. In phacoemulsification and endoscopic surgery, a small incision is made in the body and the surgical instruments are passed through the incision into the body to perform the surgical procedure. When a procedure is followed which requires a handpiece operating at a different frequency, it is necessary to withdraw the instrument being used and to replace it with another instrument with a different frequency range.

This exchange of instruments is time consuming, can cause irritation of the tissue surrounding the incision, and is a mechanism that increases the chances of introducing infections into the body. Accordingly, it would be advantageous to have a handpieces that can perform multiple functions, including those at different frequencies. Such a device could be introduced into the body through an incision, and remain there until the procedure is completed, with its function and operation frequency being changed as needed.

### SUMMARY OF THE INVENTION

In accordance with the present invention, a handpiece is provided that includes dual or more ultrasonic transducers or drivers. Advantageously, the handpiece can be an ultrasonic surgical tool. All of the current surgical ultrasonic handpieces have a single ultrasonic transducer, which limits the functioning and frequency of functioning of the handpiece.

In an illustrative embodiment, the ultrasonic vibrating element or driver (magnetostrictive or piezoelectric) surrounds the (aspiration) tube in the handpiece. When using at least a two-tube configuration at the work tip to which the irrigation and aspiration lines are directly attached, such as in the above referenced Banko patent, there is no need to have a tube through the center of the vibrating elements. Thus the vibrating elements can be much smaller in size because there is no central lumen used for aspiration. Because of these smaller dimensions, for the first time there is room for several ultrasonic vibrating elements located in parallel inside a surgical handpiece. This enables the handpiece to have two or more ultrasonic transducers or vibrating elements within its body, each of which can be independently controlled and provide vibrational energy to different connecting bodies that in turn are attached to different work tips.

In a further embodiment the two work tips are pivotally connected to each other so that the vibrations of the two independently controlled elements produces an opening and closing of the distal portions of the work tips, thus creating a clamping, sawing or cutting arrangement.

A still further embodiment has separate vibrating elements in separate housings. The work tips are connected together so that at the proximal ends the housings can be moved toward or away from each other, and at the distal end the work tips can are simultaneously moved away from and toward each other. With this arrangement the handpiece has even greater flexibility in use.

### BRIEF DESCRIPTION OF THE DRAWINGS

The foregoing and other objects and advantage of the present invention will become more apparent upon reference to the following specification and annexed drawings in which:

FIG. 1A is a plan view, partly in cross section, of one embodiment of the surgical handpiece of the present invention and FIG. 1B is a view of a rack and pinion drive for the lever of FIG. 1;

FIGS. 2A, 2B and 2C are plan views, partly in cross section, of embodiments of the surgical handpiece of the invention in which the work tips are pivotally connected so as to form a scissor, a coagulator and a bone saw, respectively;

FIG. 3 is enlarged plan view of an embodiment of the present invention with separate housings for dual vibration element, wherein the housings are pivotally connected together to form either a scissor or a coagulator.

### DETAILED DESCRIPTION OF THE INVENTION

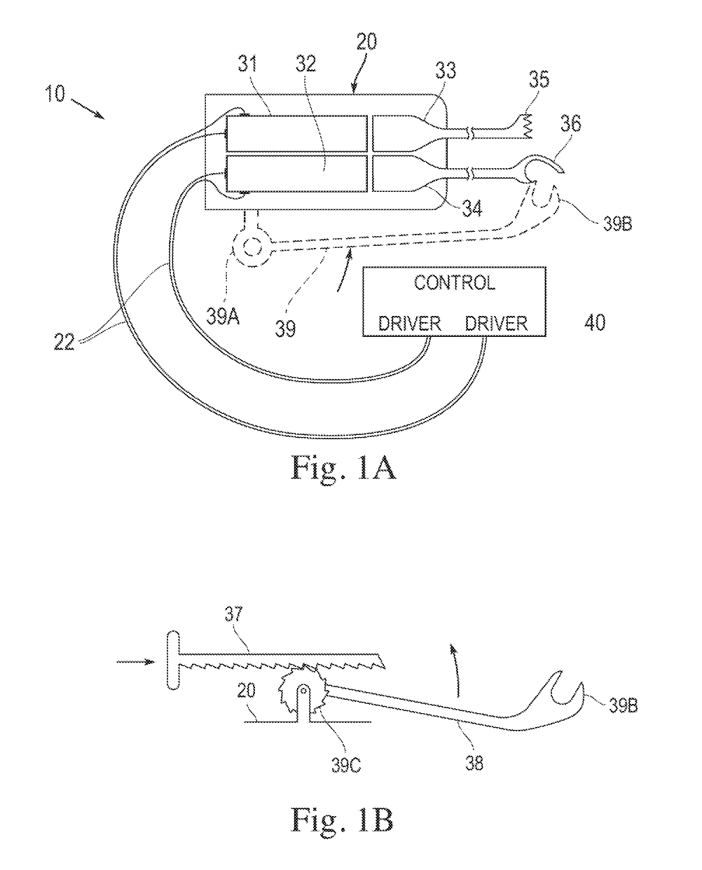

FIG. 1A shows one embodiment of the present invention where an ultrasonic handpiece 10 comprises a pair of ultrasonic drivers (or ultrasonic vibrating elements) 31, 32 located within a housing 20. A pair of connecting pieces 33, 34 is connected between the vibrating elements and a pair of working tips 35, 36, respectively. Electrical wires 22 provide electrical signal between a control unit 40 and the ultrasonic drivers 31, 32, which may be piezo electric crystals or ceramic elements. The work tips 35, 36 can be exchanged or replaced with other work tips that perform different functions.

The ultrasonic drivers 31, 32 may be magnetostrictive, having a stack of magnetostrictive laminations (not shown), or piezoelectric transducers. The control unit 40 drives the ultrasonic drivers 31, 32 so that they oscillate at an ultrasonic frequency that is typically their resonant frequency. Also, the control unit monitors the frequency of the ultrasonic drivers 31, 32 and adjusts its drive frequency to maintain an efficient operation of the stack, while keeping the ultrasonic drivers 31, 32 oscillating at their own resonant frequency. The drivers can be designed to be replaceable so that if a different operating frequency is desired, a driver with a different resonate frequency can be substituted for the existing one.

The control unit 40 is configured to adjust the output power for each of the ultrasonic drivers located within the handpiece 20. In this way, the size of the movement or displacement of each work tip can be independently controlled. This control unit 40 allows the handpiece to provide a wide variety of functions. For example, larger high frequency movements may be appropriate for breaking up the cataract during phacoemulsification, while smaller lower frequency movements may be appropriate for clean up after the cataract removal.

An irrigation line and an aspiration line (not shown) may be directly connected to each of the working tips 35, 36, where appropriate. Accordingly, even if the working tip is replaced with one having a different function, the operations of irrigation and aspiration can be properly maintained where essential, e.g., in endoscopic surgery.

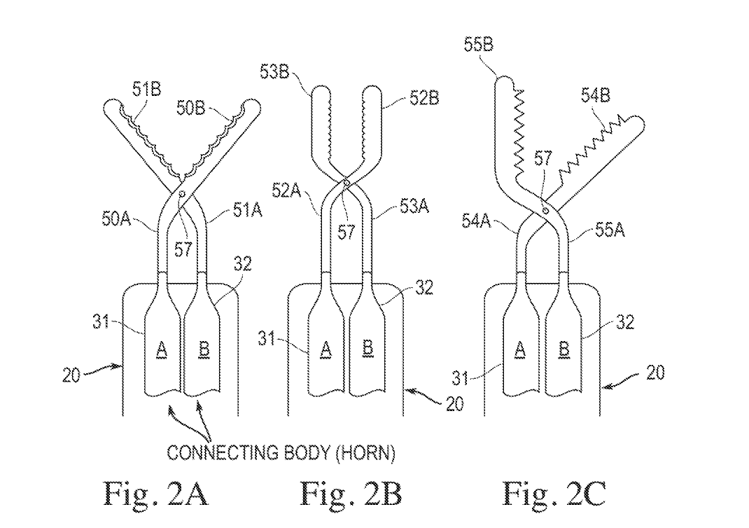

FIGS. 2A-2C indicates several embodiments where the working tips of the present invention are pivotally connected. In FIG. 2A scissor-shape working tips 50B, 51B having sharp saw tooth edges are attached to connecting pieces 50A, 51A, which in turn are attached to the ultrasonic drivers 31, 32 in the housing 20. The connecting piece/work tip 50A, 50B, 51A, 51B are pivotally connected together at pivot point 57. As an electrical signal is applied to ultrasonic drives (vibrating elements) 31, 32 the scissor work tips 50B, 51B are caused to move up and down as shown in FIG. 2A. Because of the sharp edges, the handpiece can be used as a scissor for cutting tissue when the movement of those edges is in contact with tissue.

Similarly, FIG. 2B shows a blood vessel coagulator having work tips 52B, 53B with relatively dull flat edges. The work tips are attached to connecting pieces 52A, 53A. The connecting piece/work tip 52A, 52B, 53A, 53B are pivotally connected together at pivot point 57. Like the scissor of FIG. 2A, the work tips of the coagulator are caused to move up and down as shown in FIG. 2B at an ultrasonic frequency. Because of the relatively dull edges, the handpiece can be used as a blood vessel coagulator.

As shown in FIG. 2C, saw work tips 54B, 55B, have large teeth, and are at different angles. In particular, while tip 54B is at about a 45° angle, tip 55B is nearly vertical as shown in the drawing. The work tips are connected to connecting pieces 54A, 55A, which in turn are connected to vibrating elements 31, 32. As with the other embodiments of FIGS. 2A-2C, the connecting pieces/work tips 54A, 54B, 55A, 55B are pivotally connected together at pivot point 57. The arrangement of FIG. 2C is used to cut bone or tissue.

If desired or advantageous, the pivot point may be located at the vibration null node of the connecting pieces/work tips. In such a situation the pivot point is not stressed by the vibration. In another embodiment, the junction point is at a distance from the vibration null node, which may have the effect of setting up a complex vibration pattern for the connecting piece/work tip structure.

In FIGS. 2A-2C, the pair of the working tips functions as one combined part, unlike FIG. 1A, where each of the working tips 35, 36 has a different function and thus operates independently. Ultrasonic vibration can also be used for cutting, coagulation and clamping tissues.

In another embodiment, a lever 39 (shown in dotted line) may be added to the device of FIG. 1A as needed. This lever is pivotally connected to housing 20 at 39A, so it does not vibrate but can be moved with respect to the housing. The pivotal connection at 39A can be spring loaded so the lever 39 is biased away from the housing and against a stop (not shown). The distal tip 39B of the lever 39 has a shape that will allow it to securely support tissue. In use the surgeon squeezes the lever 39 against the spring force in the direction of the arrow in FIG. 1 so that the tip approaches one of the vibrating work tips, e.g., tip 36. The tissue is held by the lever while the tip 36 acts on it, e.g., to cut the tissue. Instead of a spring loaded lever a rack 37 and pinion 39C can be used to move lever 38 against one of the work tips. In such a case pressure is applied to the end of the rack as shown by the arrow in FIG. 1B. Linear movement of the rack 37 causes rotary movement of the pinion 39C, which because the lever 38 is attached to the pinion, causes pivotal movement of the lever.

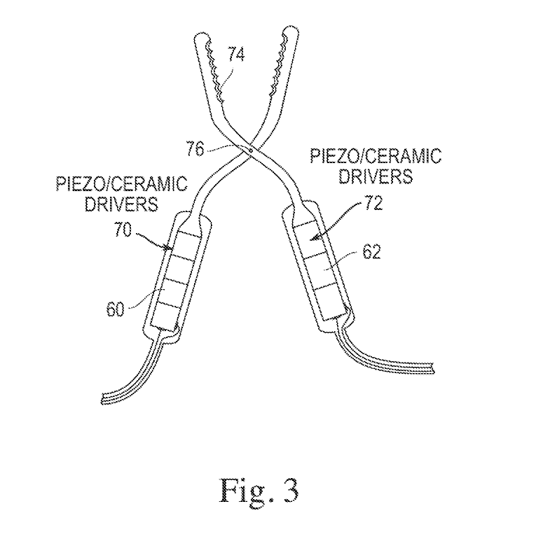

The ultrasonic handpiece device of the present invention can also have multiple ultrasonic drivers in separate housings that are connected together. FIG. 3 shows an embodiment where two separate housings 60, 62, each of which contains a single ultrasonic driver 70 or 72, are pivotally connected at a pivot point 76 just proximal of the working tips 74. If the blades of the working tips 74 have sharp edges, the working tips 74 function as a pair of scissors. If the blades of the working tips 74 have dull flat edges, the working tips 74 function as a blood vessel coagulator. These configurations provide the surgeon with more flexibility in that the work tip and housing combinations can be pivoted with respect to each other.

The combination housing and work tip joint or hinge point can be located at the null nodal point or at the non-nodal point. A null nodal point would be free from vibration. Therefore, items whose performance is adversely affected by vibration can be attached at the vibration-free nodal point. The housing and work tip combination can also be connected to a non-nodal point so there is motion. However, this requires a flexible (e.g., Teflon) joint. If a non-flexible joint is used, it changes the vibration pattern.

The present invention is not limited to the dual-driver ultrasonic handpiece. For example, more than two ultrasonic drivers can be provided in the same housing. Three or more ultrasonic drivers in the same housing provide the surgeon with more options and flexibility in an efficient manner. For instance, a coagulating operation can be performed using a working tip that has a dull surface, while performing cutting operation using another tip. Or, one ultrasonic driver can also be substituted for a spare driver in order to get a new frequency.

The multiple driver ultrasonic handpiece also eliminates the necessity of exchanging the working tips. This is particularly beneficial when limited time is available for the surgery.

The apparatus and method may also be used for many other types of surgery in other parts of the body, e.g., orthopedic, vascular and neural, such as the removal of neurological tissue. Specific features of the invention are shown in one or more of the drawings for convenience only, as each feature may be combined with other features in accordance with the invention. Alternative embodiments will be recognized by those skilled in the art and are intended to be included within the scope of the claims. Accordingly, the above description should be construed as illustrating and not limiting the scope of the invention. All such obvious changes and modifications are within the scope of the appended claims.

I claim:

1. A handpiece comprising:
at least two working tips;
at least two ultrasonic drivers each creating mechanical vibration;
at least two connecting pieces each of which connects each of one ultrasonic driver to one working tip so that the mechanical vibration of one driver is applied to one tip and the mechanical vibration of the other driver is applied to the other tip; and
a control unit that independently controls the mechanical vibration of each of the ultrasonic drivers.

2. The handpiece according to claim 1, wherein the two working tips are pivotally connected to each other at a junction point.

3. The handpiece according to claim 1, wherein the ultrasonic drivers are provided in one housing.

4. The handpiece according to claim 1, wherein the ultrasonic drivers are provided in separate housings, which are movable with respect to each other.

5. The handpiece according to claim 3 wherein the two working tips are pivotally connected to each other at a junction point.

6. The handpiece according to claim 4 wherein the two working tips are pivotally connected to each other at a junction point.

7. The handpiece of claim 6 wherein the junction point is at the vibration null node.

8. The handpiece according to claim 1, wherein the two working tips are pivotally connected to each other at a junction point and function as a pair of scissors.

9. The handpiece according to claim 1, wherein the two working tips are pivotally connected to each other at a junction point and function as a saw.

10. The handpiece according to claim 1, wherein the two working tips are pivotally connected to each other at a junction point and function as a coagulator.

11. The handpiece according to claim 1 wherein one of the working tips is a scalpel.

12. The handpiece according to claim 2, wherein the junction point is at the vibration null node.

13. The handpiece according to claim 12, wherein the junction point is at a distance from the vibration null node.

14. The handpiece according to claim 1, wherein each of the working tips is distinct and different from each other in function.

15. The handpiece according to claim 1, wherein each of the working tips is distinct and different from each other in structure.

16. The handpiece according to claim 1, further including a housing in which at least one of the drivers is located and a lever pivotally mounted on the housing that is movable toward at least one of the working tips so as to trap tissue between the lever and work tip.

17. The handpiece according to claim 1 further including a spring mechanism with as spring force that bias the lever away from the work tip, but allows the lever to move toward the work tip when the spring force is overcome.

18. The handpiece according to claim 1 further including a rack and pinion mechanism that forces the lever toward or away from the work tip as the rack is moved toward and away from the pinion.

19. The handpiece according to claim 1, wherein an irrigation line and an aspiration line are connected to each of the working tips.

20. The handpiece according to claim 1, wherein the control unit independently controls an amplitude of the mechanical vibration from each of the ultrasonic drivers.

21. The handpiece according to claim 20, wherein the ultrasonic driver can be replaced with another driver with the same or another resonant frequency so as to change the frequency of the mechanical vibration from the respective ultrasonic driver.
